(19) United States
(12) Patent Application Publication
(10) Pub. No.: US 2011/0034933 A1
(43) Pub. Date: Feb. 10, 2011

(54) LIGAMENT RECONSTRUCTION GUIDE ASSEMBLY AND METHODS OF USE

(76) Inventor: Lonnie E. Paulos, Pensacola, FL (US)

Correspondence Address: JOHN BROOKS LAW LLC, P.O. Box 156, WRENTHAM, MA, 02093, US

(21) Appl. No.: 12/937,402

(22) PCT Filed: Feb. 24, 2009
(86) PCT No.: PCT/US09/34988

§ 371 Date: Oct. 12, 2010

Related U.S. Application Data
(60) Provisional application No. 61/049,430, filed on Apr. 30, 2008.

(51) Int. Cl. A61B 17/58

(52) U.S. Cl. 606/88

(57) ABSTRACT

One embodiment of a ligament reconstruction guide assembly comprises a bracket, a cannulated guide and a reference element. These assembly elements are combined and shaped in a manner that permit a surgeon to carefully position a bone tunnel in a ligament reconstruction procedure extending from a posterior position to the knee to an anterior position relative to the knee. The bone tunnel is positioned by the reference element posterior to the knee to an anterior position relative to the knee positioned by the cannulated guide. This combination and shape allows proper positioning of a drill and tunnel and reduces the possibility of wounds to the posterior elements of the knee during tunnel creation.

### CROSS-REFERENCE TO RELATED APPLICATIONS

This application claims the benefit of U.S. App. No. 61/049,430, entitled LIGAMENT RECONSTRUCTION GUIDE ASSEMBLY AND METHODS OF USE and filed on Apr. 30, 2008, the entire contents of which is incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Technical Field

The present invention relates to an improved guide assembly to be used in the reconstruction of a torn ligament as well as methods for using the assembly. The present invention also relates to an improved ligament reconstruction guide assembly for drilling tibial and femoral bone tunnels in the reconstruction of anterior and posterior cruciate ligaments as well as methods for using the assembly.

2. Background

Guides to position bone tunnels for ligament reconstruction are known in the art. Examples of guides to create tibial tunnels for anterior curciate ligament (ACL) reconstruction include those described and disclosed in U.S. Pat. No. 6,254,605 to Howell, Stephen entitled “Tibial Guide” issued on Jul. 3, 2001 (hereinafter Howell605) and U.S. Pat. No. 5,300,077 to Howell, Stephen entitled “Methods and instruments for ACL reconstruction” issued on Apr. 5, 1994 (hereinafter Howell077) both of which are herein incorporated by reference in their entirety. These disclosures describe assemblies and methods for ACL bone tunnel creation from the front, anterior position of the knee.

Specific to posterior cruciate ligament (PCL) reconstruction procedures, it is necessary to operate on the tibia and the femoral condyl. This requires access to the front of the patient's knee and the posterior of the tibia, the back of the patient's knee. This typically requires manipulations of the patient within the operating situation to get sufficient access.

A drill guide specific for PCL reconstruction guides is disclosed in U.S. Pat. No. 4,787,377 to Jacques-Philippe Laboureau entitled “Surgical Instrument for Positiong and Insertion of Posterior Cruciate Ligament of the Knee in Plasy (or Prosthetic Replacement) (hereinafter Laboureau) which is herein incorporated by reference in its entirety. Laboureau discloses a drill guide that is passed through portals in the front of the knee and through the intercondylar notch and down the back of the tibia. Laboureau in particular is carried out from a medial parapatellar approach from the anterior position of the knee.

In other embodiments for PCL reconstruction procedures, in order to facilitate accurate placement of the guide prior to drilling the guide wire either an x-ray has to be used and/or arthroscope has to be placed through a posterior portal in the knee. These solutions expose the surgeon, patient and operating room assistants to radiation. Additionally, there is an inconvenience and risk of placing a posterior portal for arthroscopy.

### BRIEF SUMMARY OF THE INVENTION

It is an object of embodiments of the invention to provide a ligament reconstruction guide assembly for locating a tunnel through a bone for a joint ligament reconstruction comprising a reference element, a cannulated guide with a longitudinal bore having a longitudinal axis, the longitudinal axis being aligned relative to the reference element, a bracket connecting the reference element and the cannulated guide, and the bracket having a means to align the longitudinal axis relative to the reference element positioned on a bone from a posterior position to a joint.

It is a further object of embodiments of the invention to provide a ligament reconstruction guide wherein the reference element comprises a rod having a target tip and a proximal end connected to the bracket, the bracket having a curved U-shape whereby the target tip can be positioned on the bone from a posterior position to the joint and the cannulated guide can guide a bone tunnel from an anterior position to the joint.

It is another object of embodiments of the invention to provide a ligament reconstruction guide assembly wherein the assembly is capable of guiding the formation of a tibia tunnel in a posterior cruciate ligament reconstruction procedure.

It is an object of embodiments of the invention to provide a ligament reconstruction guide assembly wherein the bracket further comprises a rod bracket arm and a guide bracket arm, the rod bracket arm having a channel to slidably receive the guide bracket arm, a bracket set screw capable of securing the guide bracket arm relative to the rod bracket arm and the rod bracket arm and guide bracket arm are curved whereby the assembly is capable of positioning the target tip on a tibia from a posterior position and the cannulated guide can guide the formation of a tibia tunnel aligned with the target tip.

It is a further object of embodiments of the invention to provide a ligament reconstruction guide assembly of claim 2 wherein the bracket further comprises a rod bracket arm and a guide bracket arm, the rod bracket arm is curved in a first two-dimensional plane and the guide bracket arm is curved in the first two-dimensional plane and curved in a S-shape in a second two-dimensional plane.

It is another object of embodiments of the invention to provide a ligament reconstruction guide assembly wherein the means to align the longitudinal axis relative to the reference element comprises a slot in the guide bracket arm formed in a radial arch from a radial center of the assembly and the channel of the rod bracket arm formed in a radial arch from the radial center of the assembly whereby the slot and channel cooperate to keep the longitudinal axis of the cannulated guide and the reference element intersecting at the radial center of the assembly.

It is yet another object of embodiments of the invention to provide a ligament reconstruction guide assembly of where the curved shape of the bracket is capable of curving around a knee joint to create a tibial bone tunnel for a posterior cruciate ligament reconstruction procedure.

It is an object of embodiments of the invention to provide a ligament reconstruction guide assembly for locating a tunnel through a bone for a joint ligament reconstruction where the ligament reconstruction guide assembly comprises a means to provide a reference for positioning the creation of a bone tunnel, a means to guide a drill to create the bone tunnel and a means to align the means to guide the drill and the means to provide the reference whereby the means to provide the reference can be positioned on a bone from a posterior position to a joint.

It is an further object of embodiments of the invention to provide a ligament reconstruction guide wherein the means to provide a reference comprises a rod having a target tip and a proximal end connected to a bracket, the means to guide a drill comprises a cannulated guide having a longitudinal bore having a longitudinal axis, the longitudinal bore capable of positioning a guide wire, the cannulated guide being connected to the bracket, and the means to align the means to guide and the means to provide comprises a bracket curved in a U-shape whereby the target tip can be positioned on the bone from a posterior position to the joint and the cannulated guide can guide a bone tunnel anterior to the joint.

It is an object of embodiments of the invention to provide a method of creating a bone tunnel comprising the steps of positioning a reference element posterior on a tibia through a posterior entry portal to locate a tibia tunnel exit point, positioning a distal end of a cannulated guide on the tibia to mark a tibia tunnel entry point and drilling a tibial tunnel from the tibia tunnel entry point to the tibia tunnel exit point.

It is a further object of embodiments of the invention to provide a method of creating a bone tunnel wherein the reference element further comprises a target tip to locate the tibia tunnel exit point.

It is yet a further object of embodiments of the invention provide a method of creating a bone tunnel wherein the posterior entry portal is a posterior medial entry portal, the tibia tunnel entry point is an anterior lateral entry point on the tibia, or the tibia tunnel entry point is an anterior medial entry point on the tibia.

Is it an object of embodiments of the invention to provide a method of creating a bone tunnel further comprising the steps of positioning a trailing end of a ligament graft in the tibial tunnel, securing the trailing end of the ligament graft in the tibial tunnel, drilling a femoral tunnel in a femur, positioning a leading end of the ligament graft in the femoral tunnel and securing the leading edge of the ligament graft in the femoral tunnel.

It is another object of embodiments of the invention to provide a method of creating a bone tunnel wherein the tibial tunnel comprises two tibial tunnels capable of use with a double bundle ligament graft and the femoral tunnel comprises two femoral tunnels capable of use with a double bundle ligament graft.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a top perspective view of one embodiment of the ligament reconstruction guide assembly.

FIG. 2 illustrates a top perspective view of a partially exploded view of one embodiment of the reconstruction guide assembly.

FIG. 3 illustrates a top perspective view of one embodiment of the ligament reconstruction guide assembly showing the alignment of a cannulated guide and the reference element.

FIG. 4 illustrates a side view of one embodiment of the ligament reconstruction guide assembly.

FIG. 5A-5D illustrates a perspective view of embodiments of individual components of a ligament reconstruction guide assembly.

FIG. 6 illustrates a process diagram of one embodiment of the methods of the invention.

FIG. 7 illustrates a side view of one embodiment of the ligament reconstruction guide assembly positioned for use.

### DETAILED DESCRIPTION OF THE INVENTION

A ligament reconstruction guide assembly and methods for use will now be described in detail with reference to the accompanying drawings. It will be appreciated that although embodiments are described for use with ligament reconstruction, it is understood that the methods and systems described can be use for use in similar medical procedures where the positioning of tunnels, holes or other portals must be carefully placed. Notwithstanding the specific example embodiments set forth below, all such variations and modifications that would be envisioned by one of ordinary skill in the art are intended to fall within the scope of this disclosure.

Embodiments of this invention recognize the benefits that are available by positioning the ligament reconstruction guide assembly from a posterior position to the knee. By positioning the target point of the cannulated guide from a posterior position, it has been found that proper tunnel locations can be made more easily on the surgical table which is an environment where space and time to act are limited.

### Ligament Reconstruction Assembly

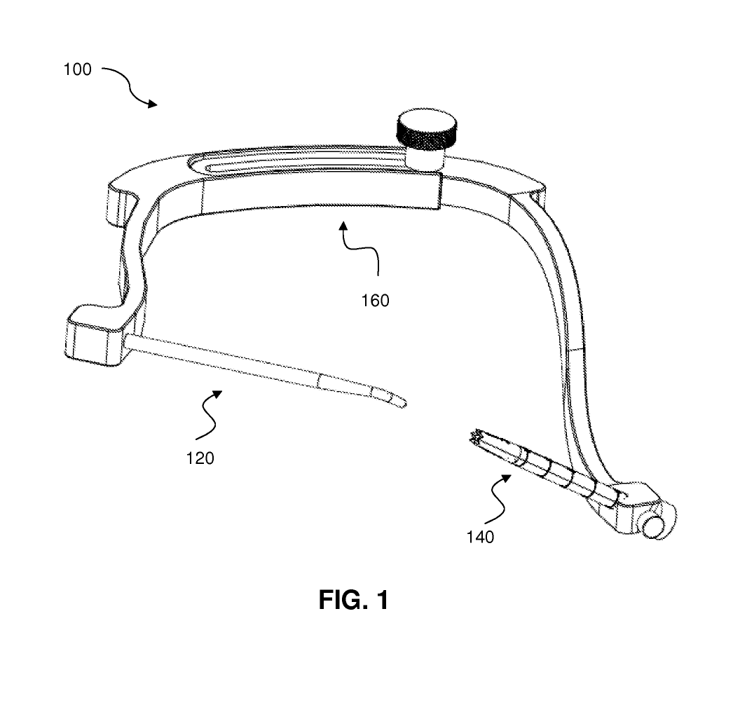

One embodiment of the ligament reconstruction guide assembly 100, as shown in FIG. 1, comprises a bracket 160, a cannulated guide 140 and a reference element 120. These assembly elements are combined and shaped in a manner that permit a surgeon to carefully position a bone tunnel in a ligament reconstruction procedure extending from a posterior position to the knee to an anterior position relative to the knee. The bone tunnel is positioned by the reference element 120 posterior to the knee to an anterior position relative to the knee positioned by the cannulated guide 140. This combination and shape allows proper positioning and reduces the possibility of wounds to the posterior elements of the knee.

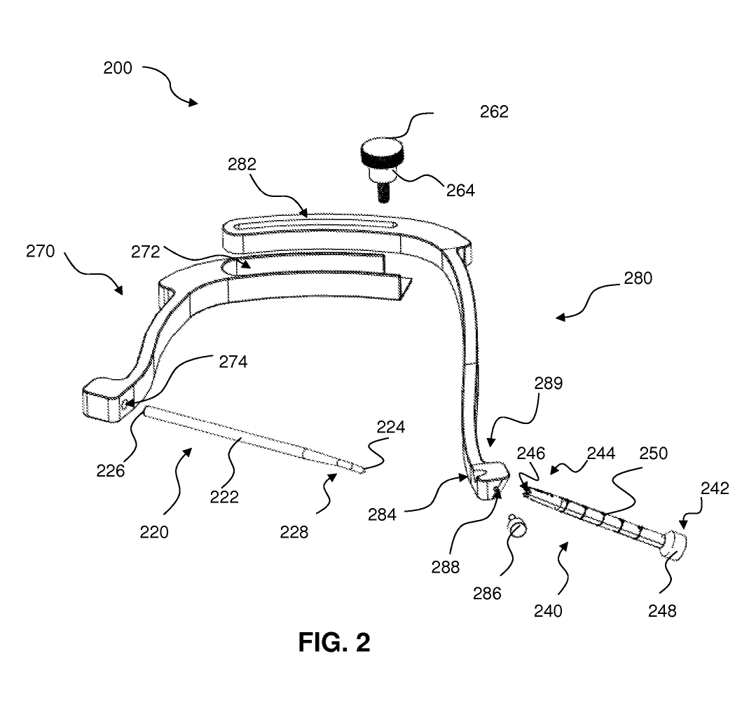

One embodiment of the bracket 160, as shown in FIG. 1, comprises a rigid U-shaped bracket connecting the cannulated guide 140 and the reference element 120. The bracket 160 made of suitable rigid surgical materials such as, but not limited to titanium, stainless steel, nitinol, metal alloys, plastics or other suitable synthetic materials. The bracket has an adjustment means to allow the bracket to move through multiple size arches and once the suitable arch is obtained, the arched bracket can be secured into that shape. In the embodiment of FIG. 2, the adjustment means comprises a rod bracket arm 270 and a guide bracket arm 280 that are slidably connected by a cooperating channel 272 that cooperatingly receives the profile of the guide bracket arm 280. The shape of the channel 272 and the bracket arm profile cooperate such that as adjustments are made to the two arms, a proper relationship is maintained between the reference element 220 and the cannulated guide 240. This proper relationship is one where a central radial point about the reference element is created and maintained within the arched bracket even when adjusted. Suitable securing means to secure the bracket arms comprises means such as a nut and bolt, pins, clips or other securing means.

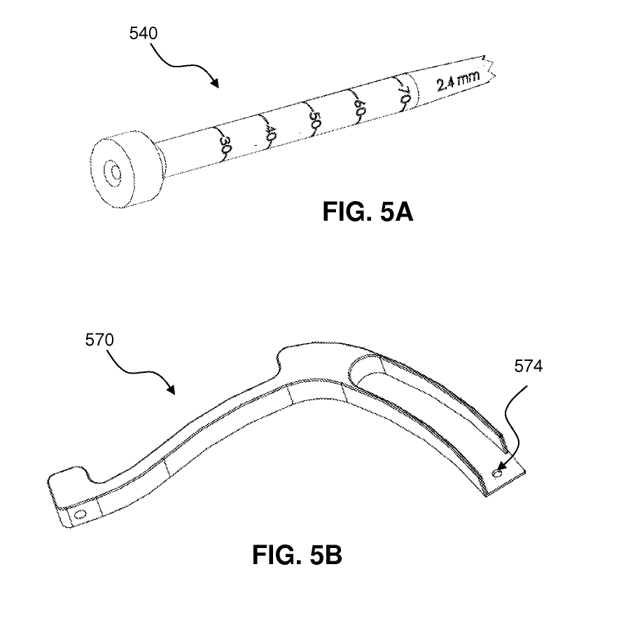

FIG. 2 also shows a threaded knob 262 as the means to secure the two bracket arms. In this embodiment, the threaded knob 262 is received in a mating threaded hole (see 574 in FIG. 5A) in the rod bracket arm 270. This knob 262 is also received through a slot 282 in the guide bracket arm 280. When the knob 262 is threaded into the threaded hole, the knob tightens on the guide bracket arm 280, a widened width 264 of the knob puts a force on the guide bracket arm 280 such that it and the rod bracket arm 270 are frictionally engaged and secured by the threaded knob 262. By unthreading the knob 262 from the bracket arms, the two bracket arms can be repositioned and they can also be frictionally engaged in this or other positions by tightening the threads on the threaded knob 262.

The rod bracket arm 270 has a connection means in its distal end to receive and connect to the distal end of the reference element. In FIG. 2, this means allows for proper alignment of the reference element and its components when the reference element is connected and ensures the proper relationship between the reference element 220 and the cannulated guide 240. In the embodiment shown, and not for limitation, the connection means is a recess 274 that allows the reference element proximal end 226 to be rigidly connected by a means such as welding. Other connection means such as mated treads, nuts and bolts, pins, clips or other connecting means are contemplated.

The guide bracket arm 280 has a connection means in its distal end to receive and connect to the cannulated guide 240. This connection means is positioned to allow proper alignment of the cannulated guide 240 with respect to the reference element 220. In the embodiment shown in FIG. 2, and not for limitation, the connection means comprises a bore 284 that allows the cannulated guide 240 to be slidably received in the distal end of the guide bracket arm 280. The connection means also includes a guide screw 286 that is received in a guide screw bore 288 to frictionally secure the cannulated guide 240 within the bore 284. Other connection means such as mated treads, nuts and bolts, pins, clips or other connecting means are contemplated.

The bore 284 in the guide bracket arm 280 is also sized to receive other elements used in ligament reconstruction procedures. These other elements include but are not limited to cannulated reamers, coring drill bits, guide pins, collars and other similar instruments used in creating bone tunnels. The bore 284 has a longitudinal axis that aligns with the longitudinal axis of the cannulated guide 240 when received and other elements to ensure proper alignment of these elements with the reference element 220. The internal size of the bore 284 is also sized to closely fit the outside dimension of the received elements to minimize movement in the bore and therefore the deviation of their longitudinal axes.

One embodiment of the reference element 220 is an elongated rigid rod 222 having a target tip 224 at its distal end. The reference element 220 is attached at its proximal end 226 to the free distal end of the rod bracket arm 270. The target tip 224 is positioned inside the radial opening of the curve of the bracket so that it is generally positioned at the central radial point of the arched bracket. Although not necessary, the embodiment in FIG. 1, the reference element 220 has a bend 228 in the rod 222 that helps the assembly maneuver the target tip 224 during use and position the target tip when positioned from a posterior medial portal during surgery.

The reference element 220 is made of a rigid surgical material such as but not limited to but not limited to titanium, stainless steel, nitinol, metal alloys, plastics or other suitable synthetic materials. The length and diameter of the reference element is sized to provide a small profile in the patients' body when used. Suitable dimensions for illustration, and not for limitation include the reference element having a length ranging from about 4 to 5 inches and more preferably about 4.5 inches and a diameter ranging from about .17 to .2 inches and more preferably .187 to .188 inches tapering to a point at its distal tip. For reference elements with a bend, the bend can be any angle that helps the user position the distal tip of the element. For illustration purposes and not for limitation, for embodiments of the reference elements with a bend, the inner angle of the bend in the reference element can be about 100-170 degrees and in one preferred embodiment, about 130 degrees.

In an embodiment of the reference element, a shield is also provided to help prevent the insertion of elements through the cannulated guide beyond the shield. The shield can be an enlarged portion of the target tip or is may be a portion of the reference element separate from the target tip such as a generally flat plate. It is also contemplated that embodiments of the assembly provide for the target tip to be positioned relative to a radial center of the assembly, and this shield may be placed at that radial center. These types of embodiments will allow positioning of the assembly with the target tip while the cannulated guide allows guide pins to be inserted and the shield prevents the guide pin from damaging tissues in the knee beyond the bone.

Referring to the embodiment in FIG. 2, the reference element 220 is connected to the rod bracket arm 270. This connection may be permanent to ensure proper alignment of the target tip 224 with the cannulated guide 240. Removable connections are also possible if they can maintain the proper alignment of the target tip 224 with the cannulated guide 240 as discussed below.

The cannulated guide 240 is a rigid elongated cylinder with proximal end 242, a distal end 244 and a longitudinally axis running down a longitudinal bore 246 of the cannulated guide. The distal end 244 of the guide 240 can be, but need not be serrated. Serrated edges help secure the cannulated guide on bone during surgery and can help bore into the bone when necessary. The proximal end 242 of the guide 240 can be, but need not be an enlarged portion 248. The longitudinal bore 246 extends through the entire length of the cannulated guide 240 and is large enough to allow surgical tools such as but not limited to guide pins, drill bits and other tools to be received through the hollow center. The length and diameter of the cannulated guide 240 are sized to provide a suitable length such that the user can position the distal end 244 of the guide on or in the patient's bone while also safely inserting a guide wire or guide pin to position the tunnel. Diameters of the longitudinal bore are those typical for surgical cannulated guides used in orthopedic procedures. Other dimensions of the cannulated guide 240 are those typical for surgical cannulated guides used in orthopedic procedures. Preferably, the cannulated guide 240 is constructed of stainless steel, although it is appreciated that any suitable surgical material may be used.

For illustration, and not for limitation, one embodiment of the cannulated guide 240 includes a diameter of the longitudinal bore 246 of about .095-.099 inches. For illustration and not for limitation, one embodiment of the cannulated guide 240 has an overall length of about 3.2-3.4 inches and an outer diameter of about .25 inches tapering towards its distal end 244. As shown in the embodiment of FIG. 1, the enlarged portion 248 has an outer diameter or about .5 inches.

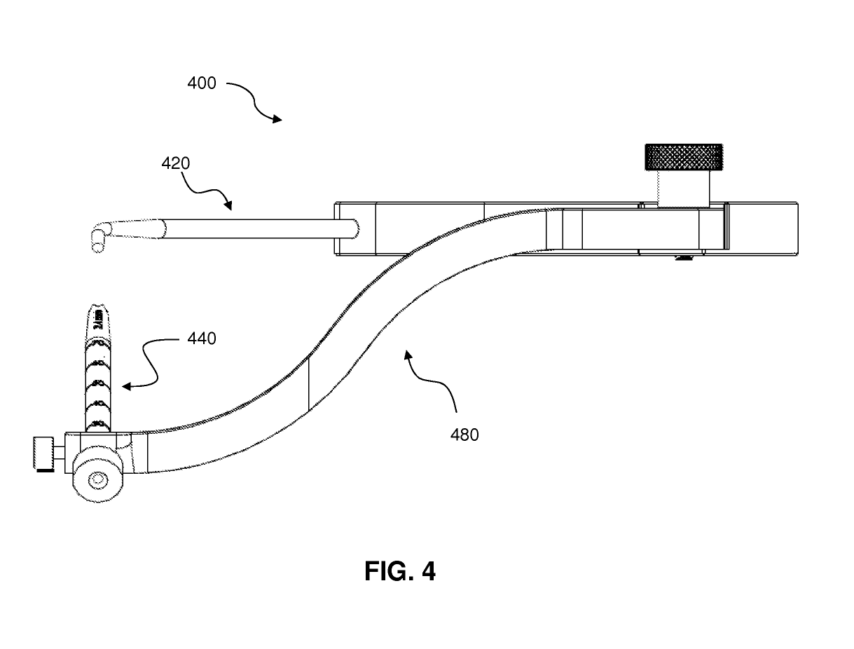

The cannulated guide 240 can be graduated with a series of calibrated markings 250 thereon. In the embodiment shown in FIG. 4, the markings 250 are in 10 mm increments and are used to determine the placement and positioning of the guide pin and the cannulated guide.

The cannulated guide 240 is removably attached to the free distal end 289 of the guide bracket arm 280 by the bracket connection means. The distal end 244 of the cannulated guide 240 is positioned towards the inner center of the bracket arch and aligned relative to the reference element 220 of the assembly 200. The attachment of the cannulated guide 240 to the guide bracket arm 280 is such that it will always allow the extended longitudinal axis of the longitudinal bore 246 to cross the radial center of the arched bracket. This extended longitudinal axis means the center line of the longitudinal bore 246 of the cannulated guide 240, whether directly within the length of the cannulated guide 240 or extending beyond its length.

One means of removably connecting the cannulated guide 240 to the guide bracket arm 280 is to have the guide fit through a bore 284 in the free end of the bracket and have a threaded guide screw 286 frictionally hold the cannulated guide 240 in place. Other attachment means are contemplated such as clips and other frictional attachment methods.

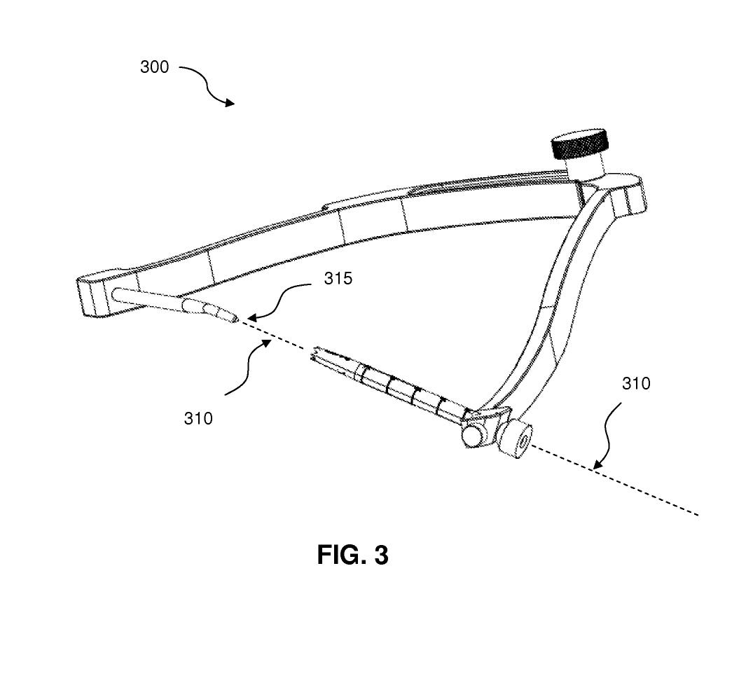

As discussed above, the shape and design of the assembly 200 provides an alignment means to ensure a proper relationship between the assembly elements. As shown in FIG. 3, when assembled, embodiments of the assembly 300 generally have a radial center. In the embodiment of FIG. 3, the radial center is the point 315 about which the elements cooperate to ensure the extended longitudinal axis of the cannulated guide is properly aligned with the reference element. The dotted line 310 represents the proper alignment of the extended longitudinal axis of the cannulated guide in relation to the target tip of the reference element. In some embodiments, the longitudinal axis is aligned to intersect directly with the tip of the reference elements. In this relationship, the target tip of the reference element is the central point of a circle and the longitudinal axis of the cannulated guide rotates about this point radially as the bracket arms are adjusted. This alignment allows the cannulated guide to guide a straight tool, such as a guide pin, a drill or a reamer, through the cannulated guide hollow center and create a bone tunnel toward the target tip. It is also contemplated that the radial center be another reference point, relative to the target tip but different from the target tip such that a tunnel can be created to that reference point. It is also understood and contemplated that similar relationships between a reference point and the longitudinal axis of the cannulated guide can be used, such as, but not limited to relationships that can be obtained through other geometric relationships such as focal points and parabolic shapes.

When assembled, the assembly also defines a gap between the reference point and the distal end of the cannulated guide. This gap is typically, but not necessarily, a gap ranging through the typical lengths of a tibial bone tunnel for a ligament reconstruction procedure. For illustration and not for limitation, one embodiment of the assembly has a gap range of about 1 to 1.5 inches or preferably about 1.2 inches. This gap can be adjusted by adjusting the position of the cannulated guide in the guide bracket arm bore.

In the embodiment illustrated in FIG. 1, the general shape of the assembly 100 is generally in a single plane. This shape allows the user to position the assembly 100 during a PCL reconstruction procedure while avoiding the obstruction caused by the patient's femur and upper leg. In this first two-dimensional plane, the bracket is curved about the radial center. In this embodiment, the assembly is also curved about a second two-dimensional plane as shown in FIG. 4 to help the user position the assembly 400 around the patient's knee. About this second plane, the guide bracket arm 480 has a s-curve shape. This s-curve can generally be at many angles that ensure proper element alignment. For illustration and not limitation, the s-curve in FIG. 4 is defined by an initial curve of about 45 degrees away from the first two-dimensional plane and a second curve of about 45 degrees back toward the first two-dimensional plane. As shown in FIG. 4, the bore 484 (not shown) in the guide bracket arm 480 is also slanted to allow the cannulated guide 440 to be properly aligned with the reference element 420. Although this embodiment has a s-curve shape and a slanted bore, it is understood that embodiments are contemplated without the s-curve or slanted bore that still keep the proper relationship between the cannulated guide and the reference element.

The embodiment shown in FIG. 5 illustrates one embodiment of the cannulated guide 540, the rod bracket arm 570 and the threaded hole 574.

It is understood that the assemblies disclosed can be modified so that they can be used in surgeries for either a left or a right knee. In one embodiment, there are both an assembly for use on a left knee and a different assembly for use on a right knee.

In these left and right embodiments for use in knee surgeries, the assembly is modified to allow for the reference element insertion from a posterior position such as a posterior medial portal.

### One Embodiment of the Methods of Use of the Ligament Reconstruction Assembly

The following description of one method of use of the ligament reconstruction assembly is to illustrate an embodiment of the methods of use for this assembly and is not intended as a limitation. It should be understood that while this invention is described in connection with particular examples and embodiments, the scope of the invention need not be so limited. Rather, those skilled in the art will appreciate that the following teachings can be used in a much wider variety of applications than the examples specifically mentioned.

One embodiment of the method is for use in arthroscopic posterior cruciate ligament (PCL) reconstruction surgery. In this embodiment, synthetic ligament grafts are provided or harvested and ligaments are reconstructed through well known methods such as those described in U.S. Pat. No. 5,300,077 and U.S. Pat. No. 6,254,605 to Howell both of which are herein incorporated by reference in their entirety. Specific to PCL reconstruction surgery, the location of tunnels and ligaments for the tibia and the femur are generally as described in U.S. Pat. No. 4,787,377 to Laboureau which is herein incorporated by reference in its entirety.

During this arthroscopic procedure, portals for the arthroscope and graft harvesting are made on the patient. Through these portals, the knee is examined by arthroscopic procedures and any observed minor defects or irregularities are taken care of.

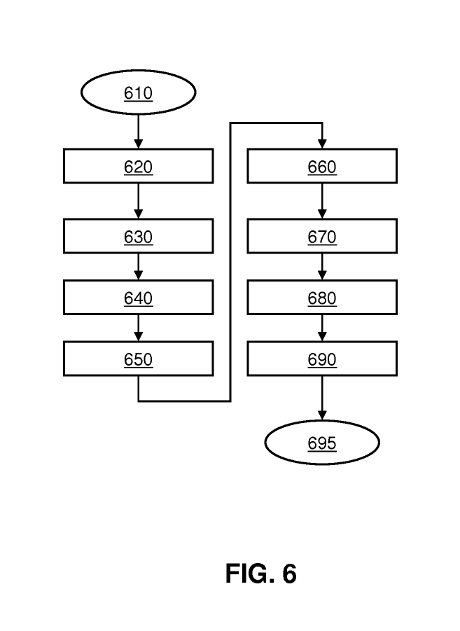

As shown in FIG. 6, one embodiment of the process 600 starts after start 610 with step 620 being positing a reference element of a ligament reconstruction guide assembly. In this embodiment, a ligament reconstruction guide assembly is provided such as shown in FIG. 1 having a bracket, reference element and a cannulated guide. This reference element of the ligament reconstruction guide assembly enters the patient's body through a posterior medial portal from the anterior knee. This entry is made by the target tip of the reference element which can be accurately placed while viewing through the intercondylar notch with the arthroscope. With the arthroscope in this position, it facilitates the surgery and allows the surgeon to clean soft tissues away from the back of the knee without jeopardizing neurovascular structures.

It is also possible to have the reference element and the target tip enter the patient's body from other posterior positions of the knee.

With the target tip of the reference element properly placed, the bracket can be adjusted by adjusting the rod bracket arm relative to the guide bracket arm to allow step 630 which is the positioning the distal end of the cannulated guide on the tibia so that the surgeon can pass a guide wire, drill or reamer through the guide.

Step 640 comprises drilling the transtibial PCL tunnel from the surgeons preferred position on the tibia to exit at the PCL anatomic insertion site as identified by the location of the target tip. With this assembly, during a PCL reconstruction operation, the surgeon can place the distal end of the cannulated guide, and therefore start the bone tunnel, from either an anterior medial or anterior lateral positions on the tibia, depending on the surgeon's preference. The means to keep the alignment of the bone tunnel with the target tip is provided by allowing the surgeon to adjust the shape of the assembly by adjusting the assembly bracket arms and securing them in the new shape with the bracket set screw. The assembly is able to enter from the posterior medial or lateral corners of the knee and can pass a guide pin or guide wire from any anterior position desired (from anterior lateral to anterior medial of the tibial tubercle) and at the same time pass the guide pin or guide wire at the correct inferior to superior angle so as to minimize neuro-vascular damage and reduce edge stress on the graft material. After placement of the entry point of the tunnel, the guide wire, drill or reamer is advanced through the cannulated guide towards the target tip. In one embodiment, the longitudinal axis of the cannulated guide is aligned with the target tip of the rigid rod. In one embodiment, a guide wire is passed by drilling or tapping through the cannulated guide to position the tunnel. If the target tip is placed at a designated exit point for a bone tunnel, the tunnel will be created and have an exit point at that point. In this mode, with the guide wire positioned in the tunnel, the cannulated guide can be removed, a collar can be placed over the guide wire and a cannulated bone boring means can be placed through the guide arm bore and over the collar and guide wire. With the bone boring means, such as but not limited to a coring drill bit, a drill bit or a reamer, aligned over the guide wire, the bone tunnel can be created with a proper alignment to the target tip.

With this method, damage to the wound to the vascular clump and neurovascular structures of the knee are protected by the target tip which prevents further entry of the wire, drill or reamer. Damage is also prevented by the surgeon's ability to visualize the exit point with the arthroscope. Because the assembly and guide pin can be passed under direct vision, it is a safe technique to help avoid damage to the neurovascular structures. It is also understood that positioning of the target tip could be done with x-ray equipment as currently used in the art.

Step 650 comprising positioning and drilling the femoral attachment site for the PCL which is visually placed and marked. The femoral lateral cortex is exposed and a bone boring means is utilized to create a small tunnel through the femur.

The surgeon can proceed to the replacement of the PCL of the knee using the medial anterior approach. Step 660 comprises passing the ligament graft through the tibia tunnel. One embodiment of this step is done by means of flexible pin connected to the leading end of the graft. When the flexible pin exits the tibia tunnel posterior, the end of the pins are grasped and pulled through the used to position and secure the trailing end of the graft in the tibia tunnel which is step 670. Once positioned in the tibia tunnel, the leading end of the graft is then inserted into the femoral tunnel as step 680. The grafts can be positioned in the tunnel through the use of the flexible pin pulling the graft through the femoral tunnel. Once the leading end is positioned it is secured in the femoral tunnel as step 690.

This embodiment of the method is completed with step 695.

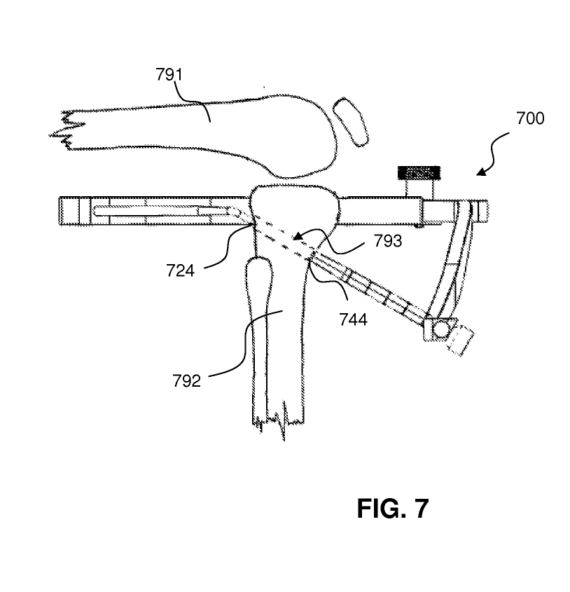

FIG. 7 shows a side view of one embodiment of the reconstruction guide 700 positioned under the femur 791 around the tibia 792 with the tibial bone tunnel 793 show from anterior to posterior aligned between the guide distal end 744 and the target tip 724.

Although the procedures described above include a single tunnel in the tibia and the femur, it is contemplated that the assembly and the methods are just as suitable for a double bundle ligament graft. This would entail multiple tunnels being created in the tibia and the femur to accommodate the multiple graft bundles.

It is also contemplated that although the procedures above include using the ligament reconstruction guide assembly to create a tibia bone tunnel, is also understood that embodiment of the guide assembly can be used to create bone tunnels in the femur and other bones.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Although this invention has been described in the above forms with a certain degree of particularity, it is understood that the present disclosure has been made only by way of example and numerous changes in the details of construction and combination and arrangement of parts may be resorted to without departing from the spirit and scope of the invention.

We claim:

1. A ligament reconstruction guide assembly for locating a tunnel through a bone for a joint ligament reconstruction, said ligament reconstruction guide assembly comprising:
a reference element;
a cannulated guide with a longitudinal bore having a longitudinal axis;
the longitudinal axis being aligned relative to the reference element;
a bracket connecting the reference element and the cannulated guide; and
the bracket having a means to align the longitudinal axis relative to the reference element positioned from a posterior position to a bone.

2. The ligament reconstruction guide of claim 1 wherein:
the reference element comprises a rod having a target tip and a proximal end connected to the bracket; and
the bracket having a curved U-shape whereby the target tip can be positioned on the bone from a posterior position to the bone and the cannulated guide can guide a bone tunnel from an anterior position to the bone.

3. The ligament reconstruction guide assembly of claim 2 wherein the assembly is capable of guiding the formation of a tibia tunnel in a posterior cruciate ligament reconstruction procedure.

4. The ligament reconstruction guide assembly of claim 2 wherein:
the bracket further comprises a rod bracket arm and a guide bracket arm;
the rod bracket arm having a channel to slidably receive the guide bracket arm; and
the rod bracket arm and guide bracket arm are curved whereby the assembly is capable of positioning the target tip on a tibia from a posterior position and the cannulated guide can guide the formation of a tibia tunnel aligned with the target tip.

5. The ligament reconstruction guide assembly of claim 2 wherein:
the bracket further comprises a rod bracket arm and a guide bracket arm;
the rod bracket arm is curved in a first two-dimensional plane; and
the guide bracket arm is curved in the first two-dimensional plane and curved in a S-shape in a second two-dimensional plane.

6. The ligament reconstruction guide assembly of claim 2 wherein the means to align the longitudinal axis relative to the reference element comprises:
a slot in the guide bracket arm formed in a radial arch from a radial center of the assembly; and
the channel of the rod bracket arm formed in a radial arch from the radial center of the assembly whereby the slot and channel cooperate to keep the longitudinal axis of the cannulated guide and the reference element intersecting at the radial center of the assembly.

7. The ligament reconstruction guide assembly of claim 2 where the curved shape of the bracket is capable of curving around a knee joint to create a tibial bone tunnel for a posterior cruciate ligament reconstruction procedure.

8. A ligament reconstruction guide assembly for locating a tunnel through a bone for a joint ligament reconstruction, said ligament reconstruction guide assembly comprising:
a means to provide a reference for positioning the creation of a bone tunnel;
a means to guide a drill to create the bone tunnel; and
a means to align the means to guide the drill and the means to provide the reference whereby the means to provide the reference can be positioned from a posterior position to a bone.

9. The ligament reconstruction guide assembly of claim 8 wherein:
the means to provide a reference comprises a rod having a target tip and a proximal end connected to a bracket;
the means to guide a drill comprises a cannulated guide having a longitudinal bore with a longitudinal axis, the longitudinal bore capable of receiving a guide wire;
the cannulated guide being connected to the bracket; and
the means to align the means to guide the drill and the means to provide the reference comprises a bracket curved in a U-shape whereby the target tip can be positioned on a bone from a posterior position to the bone and the cannulated guide can guide a bone tunnel anterior to the bone.

10. The ligament reconstruction guide assembly of claim 9 wherein the assembly is capable of guiding the formation of a tibia tunnel in a posterior cruciate ligament reconstruction procedure.

11. The ligament reconstruction guide assembly of claim 9 wherein:
the bracket further comprises a rod bracket arm and a guide bracket arm;
the rod bracket arm having a channel to slidably receive the guide bracket arm; and
the rod bracket arm and guide bracket arm are curved whereby the assembly is capable of positioning the target tip on the tibia from a posterior position and the cannulated guide can guide the formation of a tibia tunnel aligned with the rod target tip.

12. The ligament reconstruction guide assembly of claim 9 wherein:
the bracket further comprises a rod bracket arm and a guide bracket arm;
the rod bracket arm is curved in a first two-dimensional plane; and
the guide bracket arm is curved in the first two-dimensional plane and curved in a S-shape in a second two-dimensional plane.

13. The ligament reconstruction guide assembly of claim 9 wherein the means to align the means to guide and the means to provide further comprises the longitudinal axis and the reference element intersecting at a radial center of the assembly.

14. The ligament reconstruction guide assembly of claim 9 where the U-shape of the bracket is capable of curving around a knee joint to create a tibial bone tunnel for a posterior cruciate ligament reconstruction procedure.

15. A method of creating a bone tunnel comprising the steps of:
positioning a reference element posterior on a tibia through a posterior entry portal to locate a tibia tunnel exit point;
positioning a distal end of a cannulated guide on the tibia to mark a tibia tunnel entry point; and
drilling a tibial tunnel from the tibia tunnel entry point to the tibia tunnel exit point.

16. The method of claim 15 wherein the reference element further comprises a target tip to locate the tibia tunnel exit point.

17. The method of claim 15 wherein the posterior entry portal is a posterior medial entry portal.

18. The method of claim 15 wherein the tibia tunnel entry point is an anterior lateral entry point on the tibia.

19. The method of claim 15 wherein the tibia tunnel entry point is an anterior medial entry point on the tibia.

20. The method of claim 15, further comprising the steps of:
positioning a trailing end of a ligament graft in the tibial tunnel;
securing the trailing end of the ligament graft in the tibial tunnel;
drilling a femoral tunnel in a femur;
positioning a leading end of the ligament graft in the femoral tunnel; and
securing the leading edge of the ligament graft in the femoral tunnel.

21. The method of claim 15 wherein the tibial tunnel comprises two tibial tunnels capable of use with a double bundle ligament graft.

22. The method of claim 15 wherein the femoral tunnel comprises two femoral tunnels capable of use with a double bundle ligament graft.
